(19) United States
(12) Patent Application Publication
(10) Pub. No.: US 2005/0004447 A1
(43) Pub. Date: Jan. 6, 2005

(54) Flow passage selector device, and contrast medium filling tube used for the device

(76) Inventor: Tetsuya Yamamoto, Osaka-shi, Osaka (JP)

Correspondence Address: McDermott Will & Emery, 600 13th Street NW, Washington, DC, 20005-3096, US

(21) Appl. No.: 10/494,665

(22) PCT Filed: Oct. 31, 2002
(86) PCT No.: PCT/JP02/11407

(30) Foreign Application Priority Data
Nov. 5, 2001 (JP) 2001-339707
Mar. 28, 2002 (JP) 2002-91794

(51) Int. Cl. A61B 5/05

(52) U.S. Cl. 600/420

(57) ABSTRACT

A flow path switching apparatus includes a main board, and on a front side of the main board, first pinching means for deforming tube wall of a first branch tube, main pinching means for deforming tube wall of a second main tube and second pinching means for deforming tube wall of a second branch tube are provided. This structure provides a flow path switching apparatus allowing simplification of a structure of a contrast medium introducing tube that is discarded after it is used for introducing the contrast medium, and a contrast medium introducing tube to be used with the apparatus is also provided.

### TECHNICAL FIELD

The present invention relates to a flow path switching apparatus and to a structure of a contrast medium introducing tube used for the apparatus.

### BACKGROUND ART

Recently, various and many apparatuses for examining human body functions have been developed in the field of medical practice. Angiography for taking images of blood vessels to diagnose functions of humane brain or circulatory organ is one such apparatus. For taking an image of a blood vessel, a contrast medium in a syringe attached to an injector head is introduced to a patient, and an X-ray image of the contrast medium introduced to the body of the patient is taken, so as to make a diagnosis on the function of the brain or the circulatory organ.

In a cardiovascular angiography inspection, for example, when a contrast medium is to be sucked into a syringe or when blood pressure of a patient is to be monitored, it becomes necessary to switch a flow path of the contrast medium in the contrast medium introducing tube placed between the syringe and the patient. Referring to FIGS. 37 to 39, switching of the contrast medium flow path in the contrast medium introducing tube will be described.

Referring to FIG. 37, a syringe 1000 filled with contrast medium 2000 has a piston 1002 inserted therein, and a plunger 6001 attached to an injector head (not shown) is coupled to piston 1002 for moving the piston 1002.

Syringe 1000 is provided with a contrast medium inlet port 1003 and a contrast medium outlet port 1004. A contrast medium bottle is coupled to contrast medium inlet port 1003 and an automatic flow path switch 600 is coupled to contrast medium outlet port 1004.

Automatic flow path switch 600 has a cylindrical body 601 formed of a resin, containing a piston 602 and a coil spring 603 for energizing piston 602. On one side of piston 602 opposite to coil spring 603, a first coupling port 604 is provided, to which contrast medium outlet port 1004 is connected. On the barrel of body 601, a first tube 605 to be connected to the patient and a second tube 606 to be connected to the side of a pressure transducer for measuring blood pressure of the patient are provided. At the terminal end of second tube 606, a bag of physiological saline containing saline 7000 is coupled, and a roller pump 8000 is provided between pressure transducer 4000 and the bag of physiological saline.

Piston 602 has a double piston structure that includes a first piton 602a, a coupling rod 602b having one end coupled to the first piston 602a, and a second piston 602c coupled to the other end of coupling rod 602b, as shown in FIG. 38.

In the state shown in FIG. 37, piston 602 is positioned on the side of first coupling port 604, and the first and second tubes 605 and 606 may be communicated to the space between the first and second pistons 602a and 602c.

Next, the flow path switching operation using automatic flow path switch 600 having the above described structure will be briefly described.

First, in the state shown in FIG. 37, contrast medium 2000 is introduced from the contrast medium bottle to be filled in syringe 1000. When piston 1002 is pulled in the direction of the arrow by plunger 6001, contrast medium 2000 is sucked in from the contrast medium bottle through contrast medium inlet port 1003. As there is no pressure exerted on piston 602 of automatic flow path switch 600 from contrast medium outlet port 1004, the first coupling port 604 is closed by the first piston 602a. Further, as the first and second tubes 605 and 606 are in a communicable state, it is possible to measure blood pressure of the patient by pressure transducer 4000 through the first and second tubes 605 and 606. If the blood of the patient should flow back from the first tube 605 toward the second tube 606, physiological saline is fed (flushed) by roller pump 8000 toward the first tube 605, so as to stop back flow of the blood. Another object of feeding physiological saline toward the first tube 605 is to prevent possible error in the measurement of blood pressure caused by clotting of blood staying for a long period between the measurement line and a catheter, and to prevent generation of thrombus.

FIG. 39 shows a state in which contrast medium 2000 filled in syringe 1000 is introduced to the patient. When piston 1002 is pushed in the direction of the arrow by plunger 6001, pressure is applied to the first piston 602a through the first coupling port 604, and against the force of coil spring 603, piston 602 moves to the side opposite to the first coupling port 604. Consequently, the first piston 602a comes to be positioned between the first and second tubes 605 and 606, blocking communication between the first tube 605 and the second tube 606, and the flow path is switched to a state in which the first tube 605 is communicated with the first coupling port 604. As a result, contrast medium 2000 filled in syringe 1000 flows from the first coupling port 604 to the first tube 605, and introduced to the patient. At this time, no pressure is applied to the second tube 606, and therefore, pressure transducer 400 does not receive any unnecessary pressure.

In this manner, as automatic flow path switch 600 is used, an operator can be freed from troublesome manual operation of a manifold provided on conventional tubes.

Automatic flow path switch 600 described above is exchanged at every inspection, and used switch 600 will be disposed. Automatic flow path switch 600, however, consists of a large number of parts including piston 602 having a special double piston structure, and therefore, necessary cost for the switch is considerably high. This leads to increased cost for the medical institution as well as for patients. In addition, resources to be wasted must be as small as possible, in view of more efficient use of resources.

### DISCLOSURE OF THE INVENTION

An object of the present invention is to provide a flow path switching apparatus allowing a contrast medium introducing tube, which is discarded after it is used for introducing the contrast medium, to have a simple structure, and to provide the contrast medium introducing tube for use with the apparatus.

In order to attain the above described object, the present invention provides a flow path switching apparatus used with a contrast medium introducing tube including a main tube having one end coupled to an outlet port of a syringe to be filled with a contrast medium and the other end coupled to a patient side, a first branch tube branched from the main tube and coupled to a contrast medium reserving member, and a second branch tube branching from the main tube at a portion closer to the other end than the first branch tube and coupled to a pressure transducer and to a physiological saline reserving member storing physiological saline, the apparatus including: first branch tube opening/closing means for closing flow path of the first branch tube by deforming tube wall of the first branch tube and for opening the flow path of the first branch tube by recovering the tube wall of the first branch tube; main tube opening/closing means arranged between the first branch tube and the second branch tube for closing flow path of the main tube by deforming tube wall of the main tube and for opening the flow path of the main tube by recovering the tube wall of the main tube; and second branch tube opening/closing means for closing flow path of the second branch tube by deforming tube wall of the second branch tube and for opening the flow path of the second branch tube by recovering the tube wall of the second branch tube.

The contrast medium introducing tube, for use with the flow path switching apparatus having the above described structure, comes to have a structure that includes only the main tube, the first branch tube and the second branch tube. Therefore, the structure of the contrast medium introducing tube can be simplified. As a result, use of the custom-made piston having the double structure becomes unnecessary, and the cost of the contrast medium introducing tube can be reduced. Further, as the structure of the contrast medium introducing tube is simplified, air trapping in the contrast medium introducing tube can also be reduced.

In the flow path switching apparatus described above, preferably, the first branch tube opening/closing means includes first pinching means for pinching the tube path from outside to deform the tube wall of the first branch tube; the main tube opening/closing means includes main tube pinching means for pinching the tube path from outside to deform the tube wall of the main tube; and the second branch tube opening/closing means includes second pinching means for pinching the tube path from outside to deform the tube wall of the second branch tube.

As pinching means for pinching the main tube and the first and second branch tubes from outside is adopted in the flow path switching apparatus, the flow path switching apparatus is ready simply by setting the contrast medium introducing tube at a prescribed position of the apparatus, and therefore, handling is very simple.

Preferably, the flow path switching apparatus described above further includes switching means for selecting: a first state in which flow paths of the first branch tube and the second branch tube are opened by the first pinching means and the second pinching means, when the main tube is pinched by the main pinching means and the flow path of the main tube is closed; and a second state in which flow paths of the first branch tube and the second branch tube are closed by the first pinching means and the second pinching means, when the flow path of the main tube is opened by the main pinching means.

As the switching means for selecting these states is provided, when the first state is selected, the steps of discharging air in the syringe, introducing the contrast medium into the main tube and introducing the contrast medium to the patient can be performed, and when the second state is selected, the steps of sucking the contrast medium into the syringe, discharging air from the contrast medium introducing tube and re-sucking the contrast medium into the syringe can be performed. In the step of re-sucking the contrast medium, the patient side of the main tube is communicated with the second branch tube, and therefore, it becomes possible to monitor the blood pressure of the patient through the pressure transducer.

As described above, by selecting only two states, all paths can be switched. As a specific example of the switching means, a structure in which the main pinching means, the first pinching means and the second pinching means may be controlled separately and independently from each other, or a link structure for controlling the main pinching means, the first pinching means and the second pinching means in a linked manner, may be possible.

Preferably, the flow path switching apparatus described above further includes syringe holding state switching means for switching between a state in which the outlet port of the syringe is held facing approximately upward and a state in which the outlet port of the syringe is held inclined downward, with the main tube held in an approximately horizontal state.

Therefore, in the above described step of discharging air in the syringe and in the step of introducing contrast medium into the main tube, when a state in which the outlet port of the syringe is held approximately upward is selected, the air in the syringe can surely be discharged, as the air in the syringe is always driven to the outlet port.

In the step of introducing the contrast medium to the patient, a state in which the outlet port of the syringe is inclined downward is selected, so that unavoidable bubbles can be driven to the side opposite to the outlet port of the syringe, and hence, undesirable introduction of bubbles to the contrast medium introducing tube can be prevented.

In order to attain the above described object, the present invention provides a contrast medium introducing tube mounted to a flow path switching apparatus described above, the tube including: a main tube having one end coupled to a syringe to be filled with a contrast medium and the other end coupled to a patient side; a first branch tube branched from the main tube and coupled to a contrast medium reserving member; and a second branch tube branching from the main tube at a portion closer to the other end than the first branch tube and coupled to a physiological saline reserving member storing physiological saline.

Thus, the contrast medium introducing tube comes to have a structure that includes only the main tube, the first branch tube and the second branch tube. Therefore, the structure of the contrast medium introducing tube can be simplified. As a result, use of the custom-made piston having the double structure becomes unnecessary, and the cost of the contrast medium introducing tube can be reduced. Further, as the structure of the contrast medium introducing tube is simplified, air trapping in the contrast medium introducing tube can also be reduced.

Further, for the contrast medium introducing tube, preferably, a shape holding member for holding the contrast medium introducing tube in a state to be mounted to the flow path switching apparatus is further provided. As the contrast medium introducing tube is held beforehand in a state ready for attachment to the flow path switching apparatus, the contrast medium introducing tube can be attached easily and correctly to the flow path switching apparatus at one operation (one-touch). Even when the contrast medium introducing tube should be damaged and the contrast agent should be burst out, scattering of the contrast medium can be prevented because of the shape holding member, and damages to the patient, operator or medical equipment therearound can be prevented.

Preferably, in the contrast medium introducing tube, the shape holding member is provided attachable to and detachable from the flow path switching apparatus. Accordingly, dropping of the contrast medium introducing tube from the flow path switching apparatus can be prevented. Further, exchange of the contrast medium introducing tube is facilitated.

### BRIEF DESCRIPTION OF THE DRAWINGS

FIG. 1 is a first perspective view showing an overall appearance of the flow path switching apparatus and the contrast medium introducing tube in accordance with a first embodiment of the present invention.

FIG. 2 is a second perspective view showing an overall appearance of the flow path switching apparatus and the contrast medium introducing tube in accordance with the first embodiment of the present invention.

FIG. 3 is a first schematic illustration (automatic set up attitude) showing an operation of a mechanism for syringe holding state switching means in accordance with the first embodiment of the present invention.

FIG. 4 is a second schematic illustration (horizontal attitude) showing an operation of a mechanism for syringe holding state switching means in accordance with the first embodiment of the present invention.

FIG. 5 is a third schematic illustration (introducing attitude) showing an operation of a mechanism for syringe holding state switching means in accordance with the first embodiment of the present invention.

FIG. 6 is an exploded perspective view showing the structure of the first and second pinching means employed in the flow path switching apparatus in accordance with the first embodiment of the present invention.

FIG. 7 is a first schematic illustration showing the operation of the first and second pinching means employed in the flow path switching apparatus in accordance with the first embodiment of the present invention.

FIG. 8 is a second schematic illustration showing the operation of the first and second pinching means employed in the flow path switching apparatus in accordance with the first embodiment of the present invention.

FIG. 9 is an exploded perspective view showing the structure of the main pinching means employed in the flow path switching apparatus in accordance with the first embodiment of the present invention.

FIG. 10 is a first schematic illustration showing the operation of the main pinching means employed in the flow path switching apparatus in accordance with the first embodiment of the present invention.

FIG. 11 is a second schematic illustration showing the operation of the main pinching means employed in the flow path switching apparatus in accordance with the first embodiment of the present invention.

FIG. 12 represents the step of discharging air from the syringe, in accordance with the first embodiment of the present invention.

FIG. 13 represents the step of sucking the contrast medium into the syringe, in accordance with the first embodiment of the present invention.

FIG. 14 represents the step of introducing the contrast medium into the first main tube, the second main tube and the third main tube in accordance with the first embodiment of the present invention.

FIG. 15 represents the step of discharging air in the contrast medium introducing tube in accordance with the first embodiment of the present invention.

FIG. 16 represents the step of re-sucking the contrast medium into the syringe in accordance with the first embodiment of the present invention.

FIG. 17 represents the step of introducing the contrast medium into the patient, in accordance with the first embodiment of the present invention.

FIG. 18 is an exploded perspective view showing the structure of the flow path switching apparatus in accordance with a second embodiment of the present invention.

FIG. 19 is a first schematic illustration showing the operation of the flow path switching apparatus in accordance with the second embodiment of the present invention.

FIG. 20 is a second schematic illustration showing the operation of the flow path switching apparatus in accordance with the second embodiment of the present invention.

FIG. 21 is a third schematic illustration showing the operation of the flow path switching apparatus in accordance with the second embodiment of the present invention.

FIG. 22 is an exploded perspective view showing a structure of a flow path switching apparatus in accordance with a third embodiment of the present invention.

FIG. 23 is a first schematic illustration showing the operation of the flow path switching apparatus in accordance with the third embodiment of the present invention.

FIG. 24 is a second schematic illustration showing the operation of the flow path switching apparatus in accordance with the third embodiment of the present invention.

FIG. 25 is a third schematic illustration showing the operation of the flow path switching apparatus in accordance with the third embodiment of the present invention.

FIG. 26 is a first schematic illustration showing the operation of the flow path switching apparatus in accordance with a fourth embodiment of the present invention.

FIG. 27 is a second schematic illustration showing the operation of the flow path switching apparatus in accordance with the fourth embodiment of the present invention.

FIG. 28 is a third schematic illustration showing the operation of the flow path switching apparatus in accordance with the fourth embodiment of the present invention.

FIG. 29 is a perspective view representing the tube drop preventing structure and a first operation in accordance with the present invention.

FIG. 30 is a first cross sectional view representing an internal structure of the tube drop preventing structure in accordance with the present invention.

FIG. 31 is a perspective view representing a second operation of the tube drop preventing structure in accordance with the present invention.

FIG. 32 is a second cross sectional view representing an internal structure of the tube drop preventing structure in accordance with the present invention.

FIG. 33 is a perspective view representing an overall structure of a cassette.

FIG. 34 is a first schematic illustration representing an operation of attaching the cassette to the main board, viewed from the direction of the arrow A of FIG. 33.

FIG. 35 is a second schematic illustration representing an operation of attaching the cassette to the main board, viewed from the direction of the arrow A of FIG. 33.

FIG. 36 represents an arrangement in which the first and second branch tubes of the contrast medium introducing tube are directed downward.

FIG. 37 is a first illustration representing structure and operation of a conventional automatic flow path switching equipment.

FIG. 38 is a double piston structure of the conventional automatic flow path switching equipment.

FIG. 39 is a second illustration representing an operation of the conventional automatic flow path switching equipment.

### BEST MODES FOR CARRYING OUT THE INVENTION

In the following, the flow path switching apparatus and the contrast medium introducing tube in accordance with various embodiments of the present invention will be described with reference to the figures.

### First Embodiment

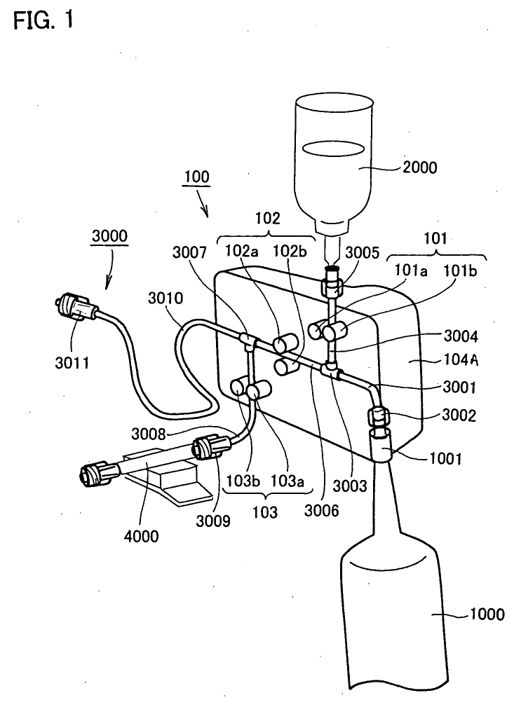

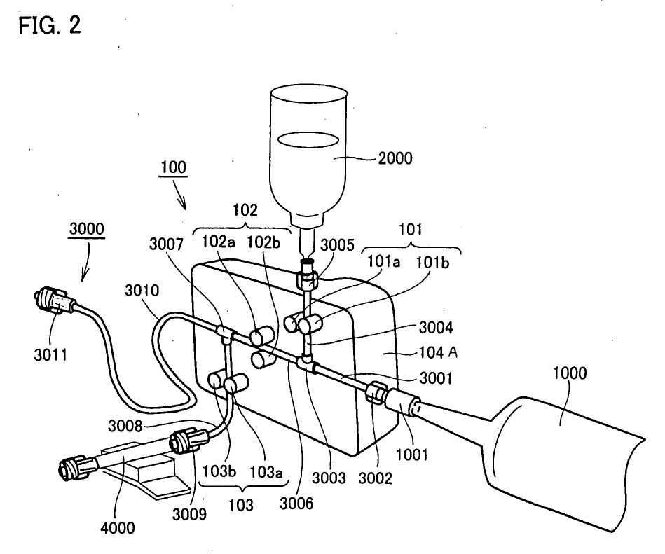

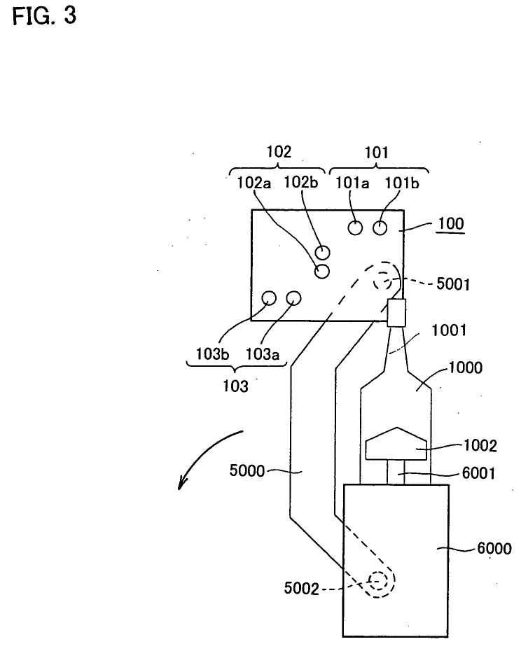

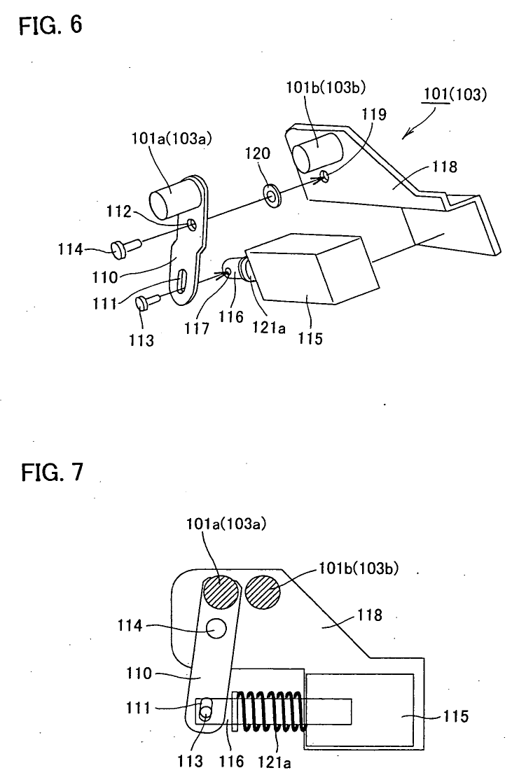

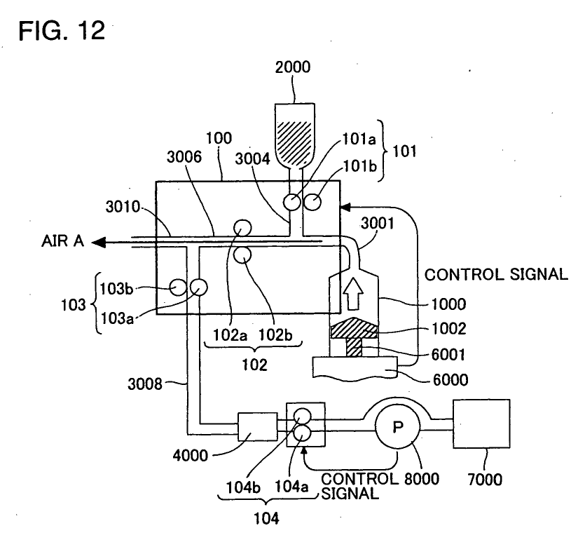

A flow path switching apparatus 100 and a contrast medium introducing tube 3000 in accordance with the first embodiment will be described with reference to FIGS. 1 to 17. FIGS. 1 and 2 are first and second perspective views showing overall appearance of flow path switching apparatus 100 and contrast medium introducing tube 3000, FIGS. 3 to 5 are first to third illustrations representing the operation of the mechanism of a syringe holding state switching means, FIGS. 6 to 11 are illustrations showing the structure of first pinching means 101, main pinching means 102 and second pinching means 103 adopted in the flow path switching apparatus 100, and FIGS. 12 to 17 show the steps of introducing a contrast medium 2000 to the patient, using flow path switching apparatus 100 and contrast medium introducing tube 3000.

(Schematic Structure of Flow Path Switching Apparatus 100 and Contrast Medium Introducing Tube 3000)

First, referring to FIGS. 1 and 2, schematic structure of flow path switching apparatus 100 and contrast medium introducing tube 3000 will be described.

First, at one end, contrast medium introducing tube 3000 is coupled to an outlet port 1001 of syringe 1000 which will be filled with the contrast medium, and at the other end, connected to a main tube, to which a patient is connected. The main tube has a first main tube 3001, a second main tube 3006 and a third main tube 3010. A female connector 3002 is provided on the side of outlet port 1001 of first main tube 3001, allowing coupling with a male connector provided in advance on outlet port 1001.

At a coupling portion between the first and second main tubes 3001 and 3006, a T-connector 3003 is arranged, and a first branch tube 3004 is provided to be branched from the first and second main tubes 3001 and 3006. At the tip end of first branch tube 3004, a male connector 3005 is provided, so that a contrast medium bottle or a syringe preparation containing a contrast medium 2000 may be coupled.

At a coupling portion between the second and third main tubes 3006 and 3010, a T-connector 3007 is arranged, and a second branch tube 3008 is provided branched from the second and third main tubes 3006 and 3010. At the tip end of second branch tube 3008, a male connector 3009 is provided, and a pressure transducer 4000 is coupled thereto. At the other end of third main tube 3010, a male connector 3011 is provided. Though not shown in FIGS. 1 and 2, a physiological saline bag containing physiological saline 7000 is coupled to pressure transducer 4000, and a roller pump 8000 is provided between pressure transducer 4000 and the physiological saline bag (see FIG. 12).

Though the first and second branching tubes 3004 and 3008 are provided extending in opposite directions, the first and second branch tubes 3004 and 3008 may be provided extending in the same direction.

A tube formed of an elastic material should preferably used for the contrast medium introducing tube 3000, as the tube wall must be deformed from the outside and must recover the original form.

Next, schematic configuration of flow path switching apparatus 100 will be described. Flow path switching apparatus 100 includes a main board 104A in which control devices are contained, and on a front surface of main board 104A, first pinching means 101 as first branch tube opening/closing means is provided, that closes flow path of first branch tube 3004 by deforming the tube wall of first branch tube 3004 and opens the flow path of first branch tube 3004 by recovering the tube wall of first branch tube 3004. As components, the first pinching means 101 has a pair of cylindrical first and second pinching members 101a and 101b, pinching from the outside the tube path of first branch tube 3004.

Further, on the front surface of the main board 104A, main pinching means 102 as main tube opening/closing means is provided, that closes the flow path of second main tube 3006 by deforming the tube wall of second main tube 3006 positioned between the first and second branch tubes 3004 and 3008 and opening the flow path of second main tube 3006 by recovering the tube wall of second main tube 3006. As components, the main pinching means 102 has a pair of cylindrical third and fourth pinching members 102a and 102b, pinching from the outside the tube path of second main tube 3006.

Further, on the front surface of main board 104A, second pinching means 103 as second branch tube opening/closing means is provided, that closes the flow path of second branch tube 3008 by deforming the tube wall of second branch tube 3008 and opening the second branch tube 3008 by recovering the tube wall of second branch tube 3008. As components, the second pinching means 103 has a pair of cylindrical fifth and sixth pinching members 103a and 103b pinching from the outside the tube path of second branch tube 3008.

In the state shown in FIG. 1, by flow path switching apparatus 100, outlet port 1001 of syringe 1000 is maintained approximately upward while the first, second and third main tubes 3001, 3006 and 3010 are held approximately horizontal, and in the state shown in FIG. 2, outlet port 1001 is inclined downward.

(Mechanism of Syringe Holding State Switching Means)

The mechanism of syringe holding state switching means added to flow path switching means 100 will be described with reference to FIGS. 3 to 5. First, referring to FIG. 3, the syringe holding state switching means has a holding lever mechanism 5000. The holding lever mechanism 5000 has one end rotatably coupled to flow path switching apparatus 100 by means of a pivot 5001, and the other end rotatably coupled to an injector head 6000 in which syringe 1000 is held, by means of a pivot 5002. A mechanism that allows rotation of injector head 6000 while keeping flow path switching apparatus 100 in a horizontal state even when flow path switching apparatus 100 is turned with respect to injector head 6000 is adopted as holding lever mechanism 5000.

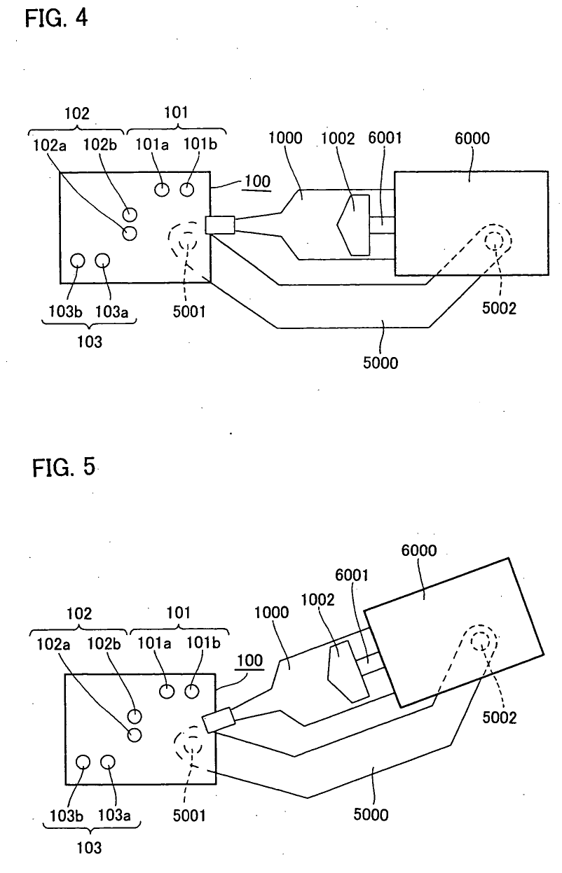

As a result, when flow path switching apparatus 100 is rotated counterclockwise from the state in which outlet port 1001 of syringe 1000 is held approximately upward (automatic set up attitude) shown in FIG. 3 about the injector head 6000, the flow path switching apparatus 100 and injector head 6000 can be set to a horizontal state (horizontal attitude shown in FIG. 4), and when flow path switching apparatus is further rotated counterclockwise, outlet port 1001 of syringe 1000 can be set to a state inclined downward (introduction attitude), as shown in FIG. 5.

(Structure of Fist Pinching Means 101, Main Pinching Means 102 and Second Pinching Means 103)

Structures of the first pinching means 101, main pinching means 102 and second pinching means 103 in accordance with the present embodiment will be described with reference to FIGS. 6 to 11. A mechanism for separately and independently control respective pinching means is adopted in the present embodiment. As will be described later, the first and second pinching means 101 and 103 are mostly closed in the normal state, and therefore, a normally closed pinching mechanism is adopted, while the main pinching means 102 is mostly opened in the normal state, and therefore, a normally open pinching mechanism is adopted.

First, the structure of first and second pinching means 101 and 103 will be described. As the first and second pinching means 101 and 103 have the same structure, only the structure of first pinching means 101 will be described. Reference characters in parentheses in the figure denote corresponding components of the second pinching means 103.

Referring to FIG. 6, the first pinching means 101 (second pinching means 103) has a cylindrical first pinching member 101a (fifth pinching member 103a) attached to one end of a lever 110. By a pin 114 and a washer 120, lever 110 is rotatably attached to base plate 118 through an axial hole 112 provided approximately at the central position of lever 110. A solenoid 115 is attached to base plate 118, and an elongate hole 111 for sliding provided at the other end of lever 110 is coupled by means of a pin 113, to a tip end portion 117 of a driving pin 116 of solenoid 115. Cylindrical second pinching member 101b (sixth pinching member 103b) is attached to base plate 118 on the side of solenoid 115 viewed from the first pinching member 101a. Further, a coil spring 121a is mounted on driving pin 116.

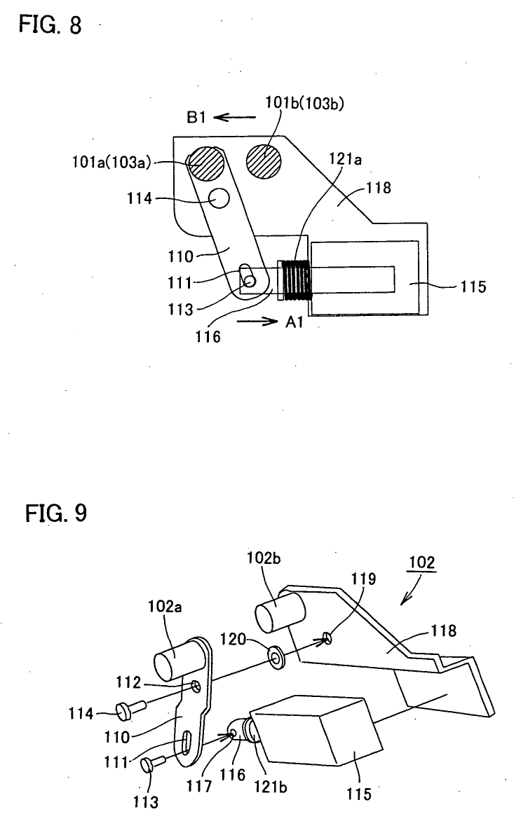

The first pinching means 101 having such a structure is normally in a closed state, that is a state in which the first and second pinching members 101a and 101b are close to each other, as shown in FIG. 7. In order to attain the open state in which the first and second pinching members 101a and 101b are apart from each other as shown in FIG. 8, solenoid 115 is turned ON so that driving pin 116 is moved in the direction of A1, lever 110 rotates about pin 114, and first pinching member 101a moves to the direction B1. When the closed state shown in FIG. 7 should be resumed from the open state shown in FIG. 8, solenoid 115 is turned OFF, so that driving pin 116 is moved back to the position shown in FIG. 7 by means of coil spring 121a.

Referring to FIG. 9, in main pinching means 102, a cylindrical third pinching member 102a is attached to one end of lever 110. Lever 110 is rotatably attached to base plate 118 by pin 114 and washer 120, through an axial hole 112 provided approximately at a central position of lever 110. A solenoid 115 is attached to base plate 118, and an elongate hole 111 for sliding provided at the other end of lever 110 is coupled by means of a pin 113, to a tip end portion 117 of a driving pin 116 of solenoid 115. A cylindrical fourth pinching member 102b is attached to base plate 118 on the opposite side of solenoid 115 viewed from the third pinching member 102a. Further, a coil spring 121a is mounted on driving pin 116.

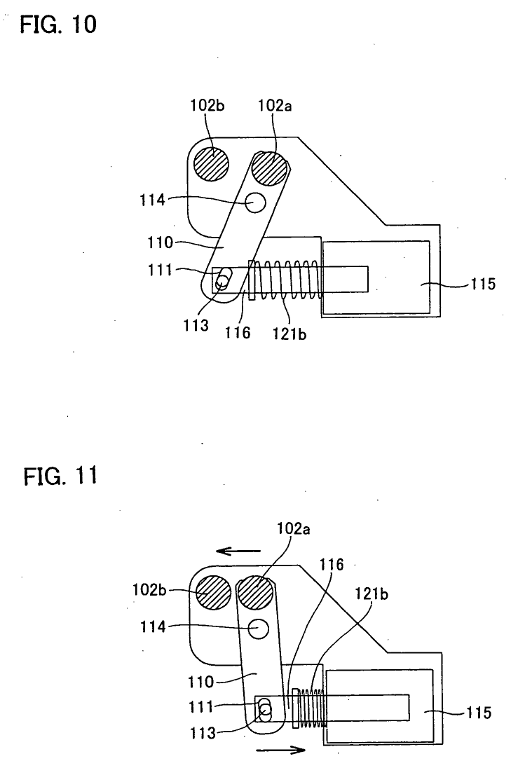

The main pinching means 102 having such a structure is normally in an open state, that is a state in which the third and fourth pinching members 102a and 102b are apart from each other, as shown in FIG. 10. In order to attain the closed state in which the third and fourth pinching members 102a and 102b are close to each other as shown in FIG. 11, solenoid 115 is turned ON so that driving pin 116 is moved in the direction of A1, lever 110 rotates about pin 114, and third pinching member 102a moves to the direction B1. When the open state shown in FIG. 10 should be resumed from the closed state shown in FIG. 11, solenoid 115 is turned OFF, so that driving pin 116 is moved back to the position shown in FIG. 10 by means of coil spring 121b.

Though solenoid 115 is used as mean for driving lever 110 in the above described structure, any other driving apparatus such as a motor (geared motor, gearless motor) may be used.

(Step of Introducing Contrast Medium 2000)

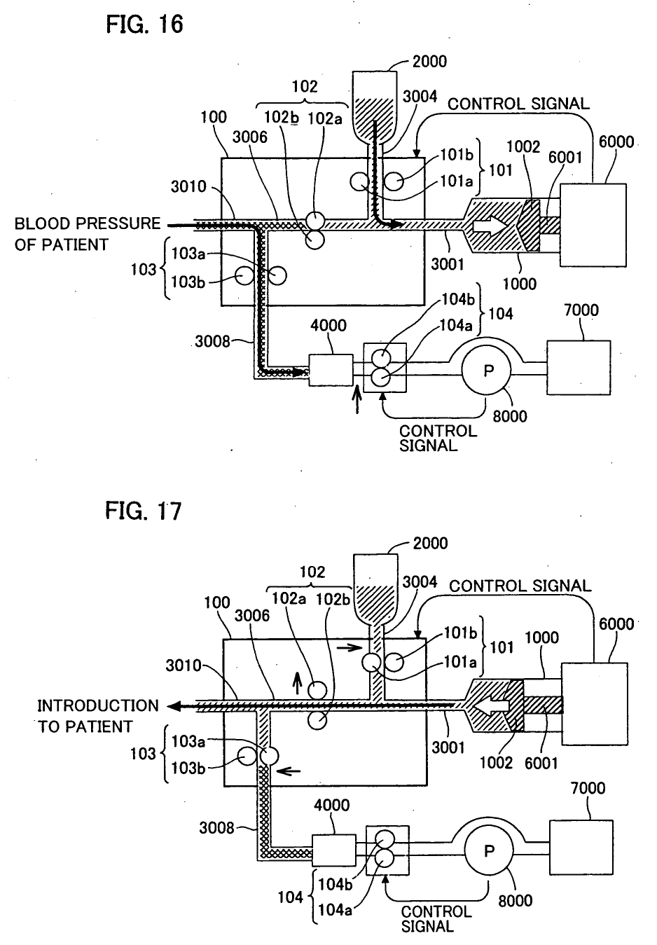

Next, referring to FIGS. 12 to 17, the step of introducing contrast medium 2000 to a patient using flow path switching apparatus 100 and contrast medium introducing tube 3000 will be described. The steps shown in FIGS. 12 to 15 are initial set up steps, and steps shown in FIGS. 16 and 17 are the steps for introducing contrast medium. Therefore, in the initial set up steps, a state in which outlet port 1001 of syringe 1000 is held approximately upward is selected, while in the contrast medium introducing steps, a state in which outlet port 1001 of syringe 1000 is kept approximately horizontal or inclined downward is selected. Switching between these states is realized by syringe holding state switching means described above with reference to FIGS. 3 to 5. In the initial set up steps, the tip end of third main tube 3010 is not connected to the side of the patient, while in the contrast medium introducing steps, the tip end of third main tube 3010 is connected to the side of the patient. It is noted that contrast medium introducing tube 3000 is set in flow path switching apparatus 100, as shown in FIGS. 1 and 2.

(Step of Discharging Air in Syringe)

First, referring to FIG. 12, the step of discharging air from the syringe 1000 will be described. First, in order to discharge air in syringe 1000 not filled with the contrast medium, piston 1002 is moved forward by plunger 6001. As plunger 6001 is controlled for this forward movement, flow path switching apparatus 100 is controlled in the following manner, by a control signal from injector head 6000. First, the first and second pinching means 101 and 103 are selected to be in the closed state, and main pinching means 102 is selected to be in the open state. Therefore, the tube paths of first and second branch tubes 3004 and 3008 are closed, and the second main tube 3006 is opened. Consequently, the first, second and third main tubes 3001, 3006 and 3010 are communicated, and from the tip end portion of third main tube 3010, the air in syringe 1000 is discharged to the outside.

(Step of Sucking Contrast Medium into Syringe)

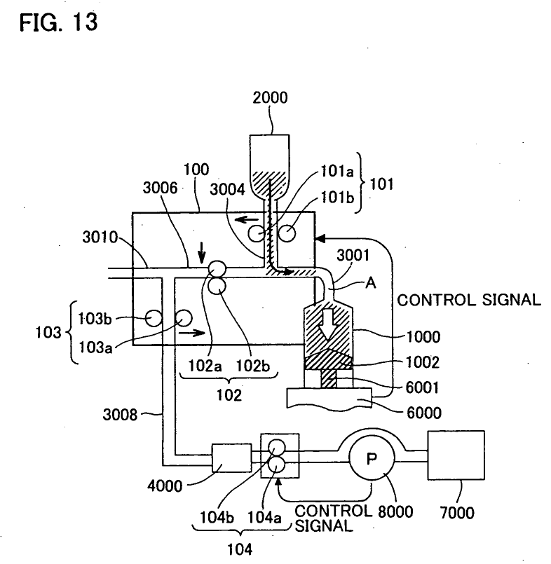

Next, referring to FIG. 13, the step of sucking contrast medium 2000 into syringe 1000 will be described. Piston 1002 is moved backward by plunger 6001. As plunger 6001 is controlled for this backward movement, flow path switching apparatus 100 is controlled in the following manner, by a control signal from injector head 6000. The first and second pinching means 101 and 103 are selected to be in the open state, and main pinching means 102 is selected to be in the closed state. Consequently, the first branch tube 3004 and the first main tube 3001 are communicated, and contrast medium 2000 is sucked and filled to syringe 1000 from a contrast medium bottle or a syringe preparation.

(Step of Introducing Contrast Medium to Main Tube)

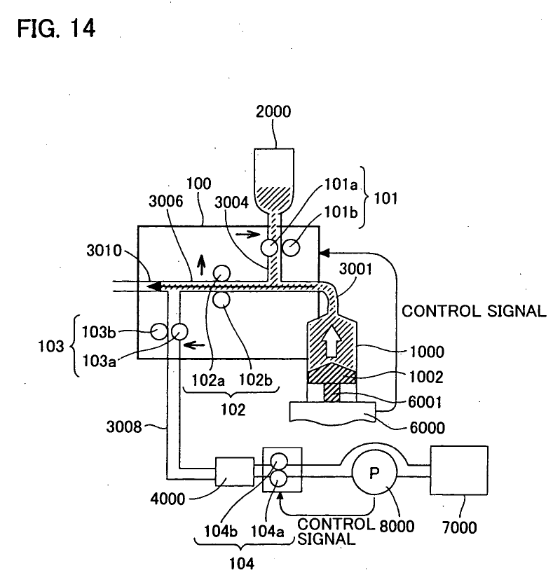

Next, referring to FIG. 14, the step of introducing contrast medium 2000 to first, second and third main tubes 3001, 3006 and 3010 will be described. Piston 1002 is moved forward by plunger 6001. As plunger 6001 is controlled for this forward movement, flow path switching apparatus 100 is controlled in the following manner, by a control signal from injector head 6000. The first and second pinching means 101 and 103 are selected to be in the closed state, and main pinching means 102 is selected to be in the open state. Consequently, tube paths of the first and second branch tubes 3004 and 3008 are closed, and the second main tube 3006 is opened. As a result, the first, second and third main tubes 3001, 3006 and 3010 are communicated, and the contrast medium 2000 in syringe 1000 is fed to the first, second and third main tubes 3001, 3006 and 3010.

(Step of Discharging Air in Contrast Medium Introducing Tube)

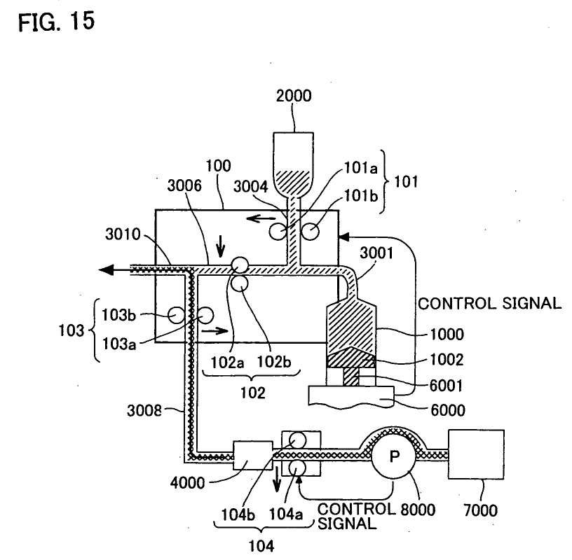

Next, referring to FIG. 15, the step of discharging air from contrast medium introducing tube 3000 will be described. Piston 1002 is selected to be in a stationary state. As plunger 6001 is controlled for this stationary state, flow path switching apparatus 100 is controlled in the following manner, by a control signal from injector head 6000. The first and second pinching means 101 and 103 are selected to be in the open state, and main pinching means 102 is selected to be in the closed state. Consequently, the first branch tube 3004 and the first main tube 3001 are communicated. Thereafter, roller pump 8000 is driven to feed physiological saline 7000 in the physiological saline bag to the first branch tube 3004 and first main tube 3001. Consequently, contrast medium introducing tube 3000 is filled either with contrast medium 2000 or physiological saline 7000, and the air in contrast medium introducing tube 3000 is completely discharged. Thus, the initial set up is complete.

(Step of Re-sucking Contrast Medium to Syringe)

Next, referring to FIG. 16, the step of re-sucking contrast medium 2000 into syringe 1000 will be described. Piston 1002 is moved backward by plunger 6001. As plunger 6001 is controlled for this backward movement, flow path switching apparatus 100 is controlled in the following manner, by a control signal from injector head 6000. The first and second pinching means 101 and 103 are selected to be in the open state, and main pinching means 102 is selected to be in the closed state. Consequently, the first branch tube 3004 and the first main tube 3001 are communicated, and contrast medium 2000 is sucked and filled to syringe 1000 from a contrast medium bottle or a syringe preparation.

(Step of Introducing Contrast Medium to Patient)

Next, referring to FIG. 17, the step of introducing contrast medium 2000 to a patient will be described. Flow path switching apparatus 100 is controlled in the following manner, by a control signal from injector head 6000. The first and second pinching means 101 and 103 are selected to be in the closed state, and main pinching means 102 is selected to be in the open state. Consequently, tube paths of the first and second branch tubes 3004 and 3008 are closed, and the second main tube 3006 is opened. As a result, the first, second and third main tubes 3001, 3006 and 3010 are communicated. Thereafter, piston 1002 is moved forward by plunger 6001. Consequently, contrast medium 2000 in syringe 1000 is introduced from the tip end portion of third main tube 3010 to the patient.

In the step of re-sucking the contrast medium described above, the second branch tube 3008 and the third main tube 3010 are communicated, and therefore, it is possible to monitor the blood pressure of the patient by pressure transducer 4000.

Between pressure transducer 4000 and roller pump 8000, a third pinching means 104 is provided for opening and closing the tube path therebetween. The third pinching means 104 includes a seventh pinching member 104a and an eighth pinching member 104b having the same structure as, for example, the first pinching means 101.

(Function and Effect)

As described above, the contrast medium introducing tube 3000, for use with the flow path switching apparatus 100 in accordance with the present embodiment, comes to have a structure that includes only the main tubes 3001, 3006, 3008, the first branch tube 3004 and the second branch tube 3008. Therefore, the structure of the contrast medium introducing tube 3000 can be simplified. As a result, use of the conventional custom-made piston having the double structure becomes unnecessary, and the cost of contrast medium introducing tube 3000 can significantly be reduced. Further, as the structure of contrast medium introducing tube 3000 is simplified, air trapping in contrast medium introducing tube 3000 can also be reduced.

As pinching means 101, 102, 103 for pinching the main tubes 3001, 3006, 3008 and the first and second branch tubes 3004, 3008 from outside are adopted in flow path switching apparatus 100, flow path switching apparatus 100 is ready simply by setting contrast medium introducing tube 3000 at a prescribed position of apparatus 100, and therefore, handling is very simple.

As the switching means for selecting open/closed states of pinching means 101, 102 and 103 is provided, in the state where the second main tube 3006 is pinched by main pinching means 102 and the flow path of second main tube 3006 is closed, when the first state is selected in which the flow paths of first branch tube 3004 and second branch tube 3008 are opened, the steps of discharging air in the syringe, introducing the contrast medium into the main tube and introducing the contrast medium to the patient can be performed.

In the state where the second main tube 3006 is opened by main pinching means 102 and the flow path of second main tube 3006 is opened, when the second state is selected in which the flow paths of first branch tube 3004 and second branch tube 3008 are closed by the first and second pinching means 101 and 103, the steps of sucking the contrast medium into the syringe, discharging air from the contrast medium introducing tube and re-sucking the contrast medium into the syringe can be performed.

In the step of re-sucking the contrast medium, the patient side of third main tube 3010 is communicated with the second branch tube 3008, and therefore, it becomes possible to monitor the blood pressure of the patient through pressure transducer 4000.

In the step of discharging air in the syringe and in the step of introducing the contrast medium into the main tube as the initial set up steps, when a state in which outlet port 1001 of syringe 1000 is held approximately upward is selected, the air in syringe 1000 can surely be discharged, as the air in syringe 1000 is always driven to outlet port 1001.

Further, in the step of introducing the contrast medium to the patient, a state in which outlet port 1001 of syringe 1000 is inclined downward is selected, so that unavoidable bubbles can be driven to the side opposite to outlet port 1001 of syringe 1000, and hence, undesirable introduction of bubbles to the contrast medium introducing tube can be prevented.

### Second Embodiment

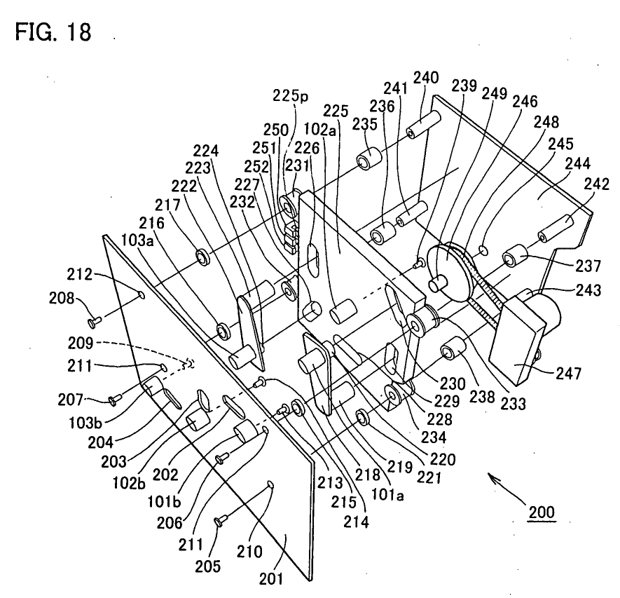

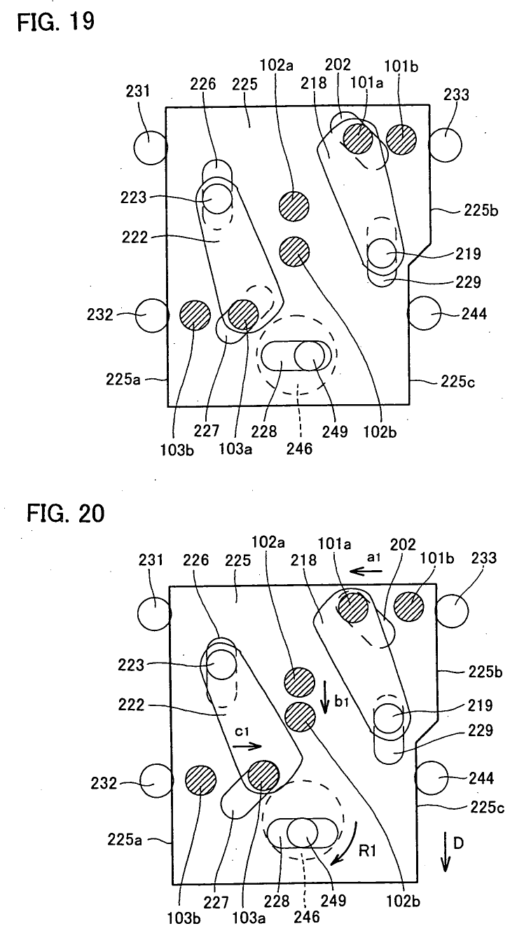

A flow path switching apparatus 200 in accordance with a second embodiment will be described with reference to FIGS. 18 to 21. In the second embodiment also, contrast medium introducing tube 3000 in accordance with the first embodiment can be used, and therefore, description of the structure of contrast medium introducing tube 3000 will not be repeated. Further, the step of introducing contrast medium 2000 using flow path switching apparatus 200 is also the same as in the first embodiment described with reference to FIGS. 12 to 17, and therefore, description thereof will not be repeated. FIG. 18 is an exploded perspective view showing a structure of flow path switching apparatus 200 in accordance with the second embodiment, and FIGS. 19 to 21 are schematic illustrations showing an operation of flow path switching apparatus 200.

Flow path switching apparatus 200 in accordance with the present embodiment is characterized in that operation for controlling each of the first pinching means 101 (first pinching member 101a, second pinching member 101b), main pinching means 102 (third pinching member 102a, fourth pinching member 102b), and second pinching means 103 (fifth pinching member 103a, sixth pinching member 103b) is realized by a link mechanism.

(Structure of Flow Path Switching Apparatus 200)

First, referring to FIG. 18, the structure of flow path switching apparatus 200 will be described. Flow path switching apparatus 200 includes a front panel 201 serving as a main board and a back panel 244. Front panel 201 and back panel 244 are fixed by using screws 205, 206, 207 and 208 such that a prescribed space is defined between front panel 201 and back panel 244 by means of spacers 240, 241, 242 and 243 provided at four corners of back panel 244. Screw holes 210, 211, 211 and 212 are opened in front panel 201 for inserting screws 205, 206, 207 and 208.

A bearing 231 for guiding a side surface portion of a driving plate 225, which will be described later, is fitted in spacer 240, and positioning spacers 217 and 235 for positioning bearing 231 are fitted, sandwiching bearing 231, in spacer 240. Similarly, a bearing 232 and positioning spacers 216 and 236 are fitted in spacer 241; a bearing 233 and positioning spacers 215 and 237 are fitted in spacer 242; and a bearing 234 and positioning spacers 221 and 238 are fitted in spacer 243.

On an upper right region of front panel 201, the second pinching member 101b is fixed by a screw 213, and on the left side of second pinching member 101b, an elongate hole 202 is formed extending in lateral direction. On the central region of front panel 201, the fourth pinching member 102b is fixed by a screw 214, and on the upper side of fourth pinching member 102b, an elongate hole 203 is formed extending in longitudinal direction. Further, on a lower left region of front panel 201, a sixth pinching member 103b is fixed by a screw 209, and on the right side of sixth pinching member 106b, an elongate hole 204 is formed extending in lateral direction.

Between front panel 201 and back panel 244, the driving plate 225 is provided, of which side surface portions are supported by bearings 231, 232, 233 and 234 to be movable upward/downward. Referring to FIG. 19, bearings 231 and 232 support a side surface 225a of driving plate 225, bearing 233 supports a side surface 225b of driving plate 225, and bearing 234 supports a recessed side surface 225c of driving plate 225. Driving plate 225 is provided with the recessed side surface 225c, so as to prevent an overrun of driving plate 225 caused by possible malfunction.

Again referring to FIG. 18, at the central portion of driving plate 225, the third pinching member 102a is fixed by screw 239 to be inserted to elongate hole 202 formed in front panel 201. On upper left side of driving plate 225, an elongate hole 203 is formed inclined downward from left to right. Below elongate hole 230 at the central portion of driving plate 225, an elongate hole 229 is formed extending in the upward/downward direction. On the upper right side of driving plate 225, an elongate hole 226 is formed extending in the upward/downward direction. On the lower left side of driving plate 225, an elongate hole 227 is formed inclined downward from right to left. At a lower central portion of driving plate 225, an elongate hole 228 is formed extending in lateral direction.

By the side of side surface 225a of driving plate 225, photo sensors 250, 251 and 252 are arranged in the upward/downward direction for detecting three positions when driving plate 252 is moved upward/downward, and on side surface 225a, a projected pin 225p is provided for intercepting an optical axis of photo sensors 250, 251 and 252.

Between front panel 201 and driving plate 225, first and second link plates 218 and 222 are arranged. On one end of the first link plate 218, the first pinching member 101a is attached, to be inserted to the elongate hole 202 formed in front panel 201. On the side of first link plate 218 opposite to first pinching member 101a, a bearing 220 is attached, that can be inserted to elongate hole 230 formed in driving plate 225. On the other end of first link plate 218, a guide pin 219 is provided to be inserted to elongate hole 229 formed in driving plate 225.

On one end of the second link plate 222, the fifth pinching member 103a is attached, to be inserted to the elongate hole 204 formed in front panel 201. On the side of second link plate 222 opposite to fifth pinching member 103a, a bearing 224 is attached, that can be inserted to elongate hole 227 formed in driving plate 225. On the other end of second link plate 222, a guide pin 223 is provided to be inserted to elongate hole 226 formed in driving plate 225.

Between driving plate 225 and back panel 244, a pulley 246 is arranged, which pulley 246 has a driving pin 249, which can be inserted to elongate hole 228 formed in driving plate 225, at a position biased from the center of rotation. An endless belt 248 is wound around pulley 246, which endless belt 248 is driven to rotate by a driving apparatus 247.

(Operation of Flow Path Switching Apparatus 200)

An operation of flow path switching apparatus 200 having the structure above will be described with reference to FIGS. 19 to 21. The state shown in FIG. 19 is a neutral state, in which contrast medium introducing tube 3000 can be attached to flow path switching apparatus 200. The first pinching means 101 (first pinching member 101a, second pinching member 101b), main pinching means 102 (third pinching member 102a, fourth pinching member 102b) and second pinching member 103 (fifth pinching member 103a, sixth pinching member 103b) are all in the open state.

FIG. 20 shows a state in which driving pin 249 is rotated clockwise by 45° (direction of the arrow R1 in the figure). By the rotation of driving pin 249, driving plate 225 is moved downward (direction of the arrow D in the figure) by a prescribed distance. Consequently, the first pinching member 101a moves in the direction of the arrow a1 in the figure along elongate hole 202. As a result, the first pinching means 101 is opened. The third pinching member 102a moves downward (in the direction of the arrow b1 in the figure) together with driving plate 225. As a result, main pinching means 102 is closed. Further, the fifth pinching member 103a is moved in the direction of the arrow c1 in the figure, along elongate hole 227. As a result, the second pinching means 103 is opened.

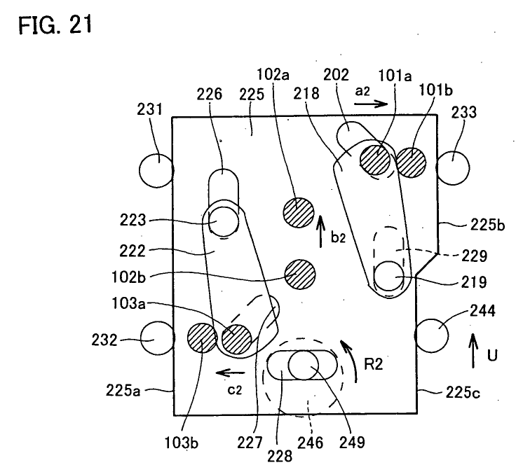

FIG. 21 shows a state in which driving pin 249 is rotated counterclockwise (in the direction of the arrow R1 in the figure) by 45° from the neutral state of FIG. 19. By the rotation of driving pin 249, driving plate 225 moves upward (in the direction of the arrow U in the figure) by a prescribed distance. Consequently, the first pinching member 101a moves in the direction of the arrow a2 in the figure along elongate hole 202. As a result, the first pinching means 101 is closed. The third pinching member 102a moves upward (in the direction of the arrow b2 in the figure) together with driving plate 225. As a result, main pinching means 102 is opened. Further, the fifth pinching member 103a moves in the direction of the arrow c2 in the figure along elongate hole 227. As a result, the second pinching means 103 is closed.

(Function and Effect)

By the present embodiment, functions and effects similar to that of the first embodiment can be attained. Further, opening and closing operations of the first pinching means 101 (first pinching member 101a, second pinching member 101b), main pinching means 102 (third pinching member 102a, fourth pinching member 102b) and second pinching means 103 (fifth pinching member 103a, sixth pinching member 103b) can be controlled linked to a sliding movement of driving plate 225 in upward/downward directions. Further, simply by selecting upward or downward movement of driving plate 225, flow path switching of all pinching means is possible.

Further, as a mechanism for sliding driving plate 225 upward/downward, position of main pinching means 102 positioned at the center may be arbitrarily selected along the direction of extension of second main tube 3006. As a result, it becomes possible, by way of example, to arrange main pinching means 102 close to pressure transducer 4000.

Though a mechanism using a pulley is adopted as a mechanism for sliding driving plate 225 upward/downward, the mechanism is not limited thereto, and a mechanism using a wire may be adopted to realize sliding movement upward/downward by a wire operation.

### Third Embodiment

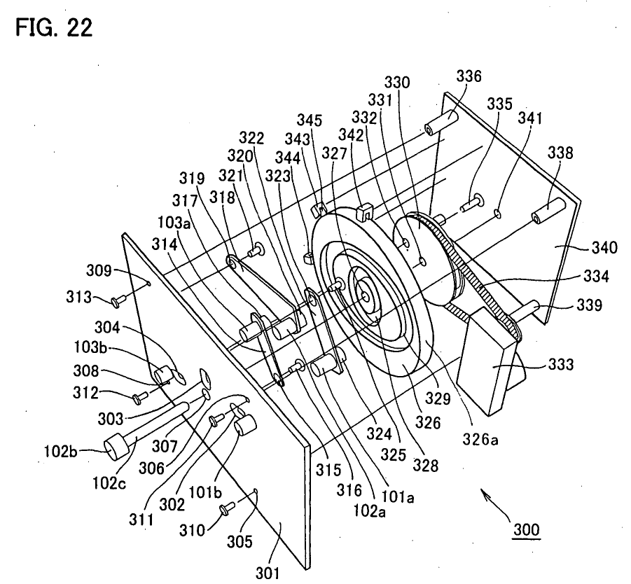

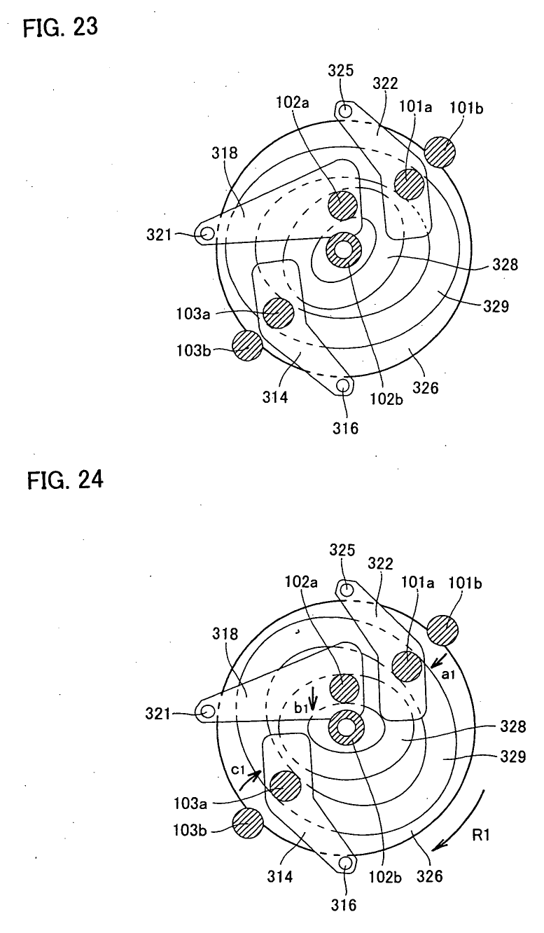

A flow path switching apparatus 300 in accordance with a third embodiment will be described with reference to FIGS. 22 to 25. In the third embodiment also, contrast medium introducing tube 3000 in accordance with the first embodiment can be used, and therefore, description of the structure of contrast medium introducing tube 3000 will not be repeated. Further, the step of introducing contrast medium 2000 using flow path switching apparatus 300 is also the same as in the first embodiment described with reference to FIGS. 12 to 17, and therefore, description thereof will not be repeated. FIG. 22 is an exploded perspective view showing a structure of flow path switching apparatus 300 in accordance with the third embodiment, and FIGS. 23 to 25 are schematic illustrations showing an operation of flow path switching apparatus 300.

Similar to the second embodiment, flow path switching apparatus 300 in accordance with the present embodiment is characterized in that operation for controlling each of the first pinching means 101 (first pinching member 101a, second pinching member 101b), main pinching means 102 (third pinching member 102a, fourth pinching member 102b), and second pinching means 103 (fifth pinching member 103a, sixth pinching member 103b) is realized by a link mechanism.

(Structure of Flow Path Switching Apparatus 300)

Referring to FIG. 22, the structure of flow path switching apparatus 300 will be described. Flow path switching apparatus 300 includes a front panel 301 serving as a main board and a back panel 340. Front panel 301 and back panel 340 are fixed by using screws 310, 311, 312 and 313 such that a prescribed space is defined between front panel 301 and back panel 340 by means of spacers 336, 337, 338 and 339 provided at four corners of back panel 340. Screw holes 305, 306, 308 and 309 are opened in front panel 301 for inserting screws 310, 311, 312, and 313.

On the upper right region of front panel 201, second pinching member 101b is fixed by a screw or the like, and on the left side of second pinching member 101b, an elongate hole 302 is formed extending in lateral direction. At a central region of front panel 301, a through hole 307 is formed, to which an axial pin 102c provided on fourth pinching member 102b is inserted, so as to allow arrangement of fourth pinching member 102b. Above through hole 307, an elongate hole 303 is formed extending in upward/downward direction. Further, on the lower left region of front panel 201, sixth pinching member 103b is fixed by a screw or the like, and on the right side of sixth pinching member 106b, an elongate hole 304 is formed extending in the lateral direction.

Between front panel 301 and back panel 340, a driving plate 326 is provided that rotates about an axial pin 102c. At the central portion of driving plate 225, a central hole 327 is formed, through which axial pin 102c is inserted. A first guide groove 328 having a prescribed elliptical orbit extending long in upward/downward direction is formed outside the central hole 327. Further, a second guide groove 329 having a prescribed elliptical orbit extending long in the lateral direction orthogonally crossing the upward/downward direction is provided outside the first guide groove 328.

By the side of side surface of driving plate 326, photo sensors 342, 343 and 344 are provided circumferentially for detecting three positions when driving plate 326 is rotated, and on side surface 326a, a projected pin 345 is provided for intercepting an optical axis of photo sensors 342, 343 and 344.

Between front panel 301 and driving plate 326, first, second and third link plates 322, 318 and 314 are arranged. On one end of first link plate 322, the first pinching member 101a is attached, to be inserted to elongate hole 302 formed in front panel 301. On the first link plate 322 opposite to the side of first pinching member 101a, a bearing 324 is attached, that can be inserted to the second guide groove 329 formed in driving plate 326. On the other end of first link plate 322, an axial hole 323 is provided, for rotatable attachment to front panel 301 by means of a pin 325.

On one end of second link plate 318, the third pinching member 102a is attached, to be inserted to elongate hole 303 formed in front panel 301. On the second link plate 318 opposite to the side of third pinching member 102a, a bearing 320 is attached, that can be inserted to the first guide groove 328 formed in driving plate 326. On the other end of second link plate 318, an axial hole 319 is provided, for rotatable attachment to front panel 301 by means of a pin 321.

On one end of third link plate 314, fifth pinching member 103a is attached to be inserted to elongate hole 304 formed in front panel 301. On the third link plate 314 opposite to the side of fifth pinching member 103a, a bearing 317 is attached, that can be inserted to the second guide groove 329 formed in driving plate 326. On the other end of third link plate 314, an axial hole 315 is provided, for rotatable attachment to front panel 301 by means of a pin 316.

Between driving plate 326 and back panel 340, a pulley 330 is arranged, and endless belt 334 is wound around pulley 326. Endless belt 334 is driven to rotate by a driving apparatus 333. An axial pin 102c having an axial hole 331 passes through the center of rotation of pulley 326. An end portion of axial pin 102c is supported by an axial hole 341 formed on back panel 340. As driving plate 326 and pulley 330 must rotate in synchronization, a pin 335 is inserted to driving plate 326, through a through hole 332 formed in pulley 330.

(Operation of Flow Path Switching Apparatus 300)

The operation of flow path switching apparatus 300 having the structure above will be described with reference to FIGS. 23 to 25. The state shown in FIG. 23 is a neutral state, in which contrast medium introducing tube 3000 can be attached to flow path switching apparatus 200. The first pinching means 101 (first pinching member 101a, second pinching member 101b), main pinching means 102 (third pinching member 102a, fourth pinching member 102b) and second pinching means 103 (fifth pinching member 103a, sixth pinching member 103b) are all in the open state.

FIG. 24 shows a state in which driving plate 326 is rotated clockwise (in the direction of the arrow R1 in the figure) by 45°. By the rotation of driving plate 326, the first pinching member 101a is moved in the direction of the arrow al in the figure along the second guide groove 329. As a result, the first pinching means 101 is opened. The third pinching member 102a moves in the direction of the arrow b1 in the figure along the first guide groove 328. As a result, main pinching means 102 is closed. Further, the fifth pinching means 103a moves in the direction of c1 in the figure along the second guide groove 329. As a result, the second pinching means 103 is opened.

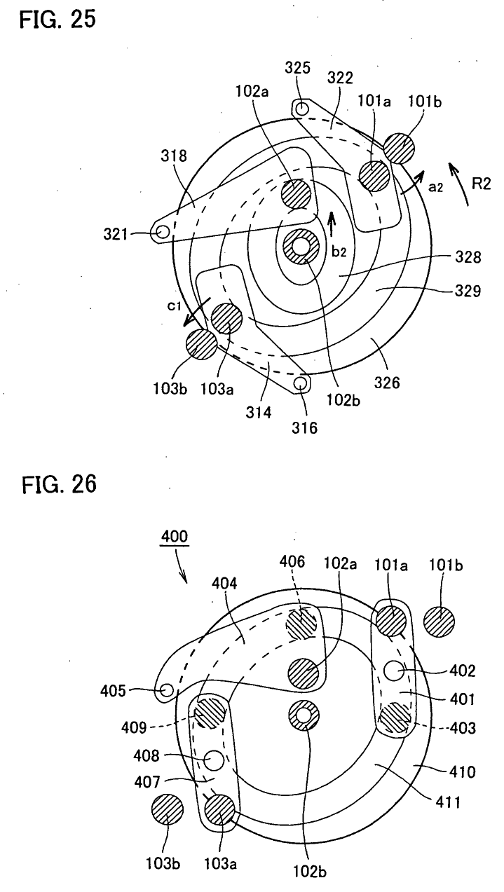

FIG. 25 shows a state in which driving pin 326 is rotated counterclockwise (in the direction of the arrow R2 in the figure) by 45° from the neutral state of FIG. 23. Because of the rotation of driving plate 326, the first pinching member 101a moves in the direction of the arrow a2 in the figure along the second guide groove 329. As a result, the first pinching means 101 is closed. The third pinching member 102a moves in the direction of the arrow b2 in the figure along the first guide groove 328. As a result, main pinching means 102 is opened. Further, the fifth pinching member 103a moves in the direction of the arrow c2 along the second guide groove 329. As a result, the second pinching means 103 is closed.

(Function and Effect)

By the present embodiment, functions and effects similar to that of the first embodiment can be attained. Further, in the present embodiment, opening and closing operations of the first pinching means 101 (first pinching member 101a, second pinching member 101b), main pinching means 102 (third pinching member 102a, fourth pinching member 102b) and second pinching means 103 (fifth pinching member 103a, sixth pinching member 103b) can be controlled linked to a rotating movement of driving plate 326. Further, simply by selecting direction of rotation of driving plate 326, flow path switching of all pinching means is possible.

As two independent groove structures of the first and second guide grooves 328 and 329 are used, the timing of opening and closing the first and fifth pinching members 101a and 103a and that of the third pinching member 102a can be adjusted independent from each other. As a result, the timing of opening and closing the first and second pinching means 101 and 103 and that of the main pinching means 102 can be finely adjusted. Therefore, it becomes possible to temporarily set the first, second and main pinching means 101, 103 and 102 all to the closed state.

### Fourth Embodiment

A flow path switching apparatus 400 in accordance with a fourth embodiment will be described in the following with reference to FIGS. 26 to 28. The basic structure of the fourth embodiment is the same as that of the third embodiment described above. Different from the third embodiment in which the first and second guide grooves 328 and 329 are provided on driving plate 326, in the present embodiment, only one similar groove is provided on the driving plate. Therefore, only the driving plate and the link mechanism thereof will be described here.

(Structure of Flow Path Switching Apparatus 400)

First, referring to FIG. 26, the structure of flow path switching apparatus 400 will be described. A driving plate 410 is provided, which has an elliptical guide groove 411 that is inclines by 45°.

On an upper right region of driving plate 410, a first link plate 401 is provided, which rotates about an axis of rotation 402 (fixed on the front panel). On one end of first link plate 401, the first pinching member 101a is attached. On the other end of first link plate 401 opposite to the first pinching member 101a, a bearing 403 is attached, which can be inserted to a guide groove 411 provided on driving plate 410.

On an upper central region of driving plate 410, a second link plate 404 is provided, which rotates about an axis of rotation 405 (fixed on the front panel). On one end of second link plate 404, the third pinching member 102a is attached. On the other end of second link plate 404 opposite to the third pinching member 102a, a bearing 406 is attached, which can be inserted to guide groove 411 provided on driving plate 410.

On a lower left region of driving plate 410, a third link plate 407 is provided, which rotates about an axis of rotation 408 (fixed on the front panel). On one end of third link plate 407, the fifth pinching member 103a is attached. On the other end of third link plate 407 opposite to the fifth pinching member 103a, a bearing 409 is attached, which can be inserted to a guide groove 411 provided on driving plate 410.

(Operation of Flow Path Switching Apparatus 400)

Next, the operation of flow path switching apparatus 400 having the structure above will be described with reference to FIGS. 26 to 28. The state shown in FIG. 26 is a neutral state, in which contrast medium introducing tube 3000 can be attached to flow path switching apparatus 400. The first pinching means 101 (first pinching member 101a, second pinching member 101b), main pinching means 102 (third pinching member 102a, fourth pinching member 102b) and second pinching member 103 (fifth pinching member 103a, sixth pinching member 103b) are all in the open state.

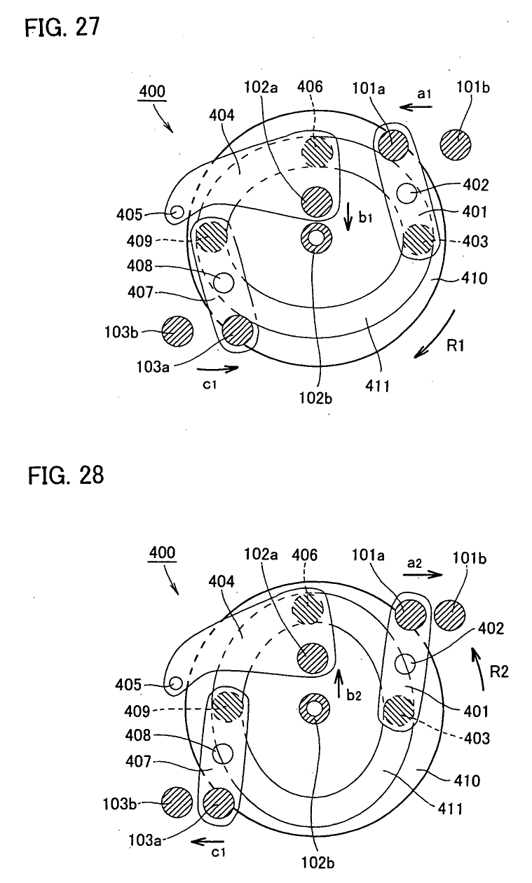

FIG. 27 shows a state in which driving plate 410 is rotated clockwise (in the direction of the arrow R1 in the figure) by 45°. By the rotation of driving plate 410, bearing 403 moves along guide groove 411, and the first pinching member 101a moves in the direction of the arrow a1 in the figure. As a result, the first pinching means 101 is opened. Bearing 406 moves along guide groove 411, and the third pinching member 102a moves in the direction of the arrow b1 in the figure. As a result, main pinching means 102 is closed. Further, bearing 409 moves along guide groove 411, and the fifth pinching member 103a moves in the direction of the arrow c1 in the figure. As a result, the second pinching means 103 is opened.

FIG. 28 shows a state in which driving pin 410 is rotated counterclockwise (in the direction of the arrow R2 in the figure) by 45° from the neutral state of FIG. 26. Because of the rotation of driving plate 410, bearing 403 moves along guide groove 411, and the first pinching member 101a moves in the direction of the arrow a2 in the figure. As a result, the first pinching means 101 is closed. Bearing 406 moves along guide groove 411, and the third pinching member 102a moves in the direction of the arrow b2 in the figure. As a result, main pinching means 102 is opened. Further, bearing 409 moves along guide groove 411, and the fifth pinching member 103a moves in the direction of the arrow c2 in the figure. As a result, the second pinching means 103 is closed.

(Function and Effect)

By the present embodiment also, functions and effects similar to that of the first embodiment can be attained. Further, in the present embodiment, opening and closing operations of the first pinching means 101 (first pinching member 101a, second pinching member 101b), main pinching means 102 (third pinching member 102a, fourth pinching member 102b) and second pinching means 103 (fifth pinching member 103a, sixth pinching member 103b) can be controlled linked to a rotating movement of driving plate 326. Further, simply by selecting direction of rotation of driving plate 410, flow path switching of all pinching means is possible.

Further, as a structure in which only one groove is formed on driving plate 326 is adopted, the overall structure can be simplified as compared with the structures of the second and third embodiments.

In the second and third embodiments, the guide grooves provided on the driving plate are continuous grooves. This is to prevent damage to the link plate or the like even when it becomes impossible to stop rotation of the driving plate by malfunction. From the view point of implementing the present invention, the guide groove may be formed only over the range of movement of the bearing, and continuous groove is not necessary.

(Mechanism for Preventing Tube Dropping)

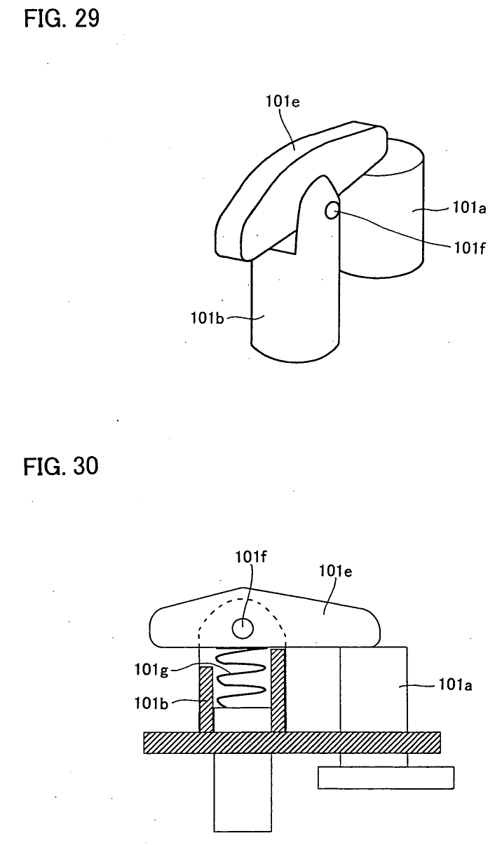

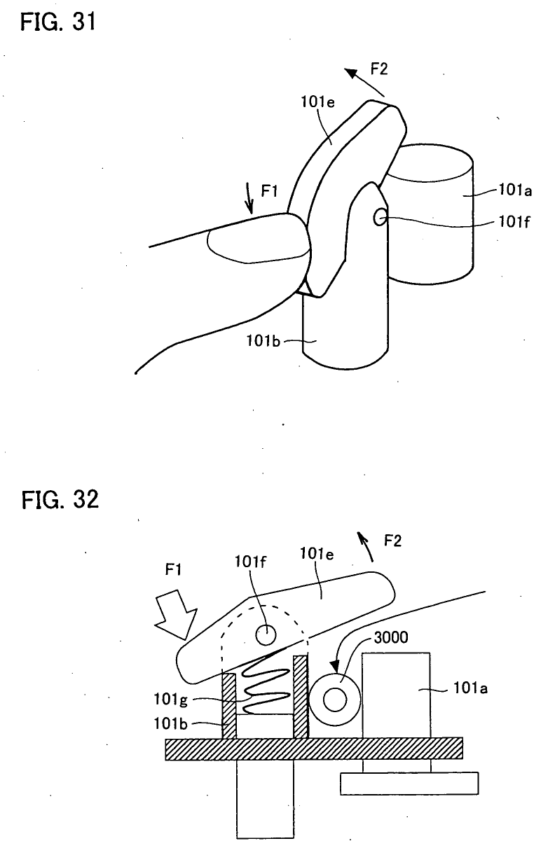

In the embodiments above, a mechanism for preventing dropping of contrast medium introducing tube 3000 may be provided at the first pinching member 101a, second pinching member 101b, third pinching member 102a, fourth pinching member 102b, fifth pinching member 103 a and sixth pinching member 103b. The mechanism for preventing dropping of the tube will be described with reference to FIGS. 29 to 32. The mechanism for preventing dropping of the tube may preferably be provided on the second, fourth and sixth pinching members 101b, 102b and 103b, and by way of example, one provided on the second pinching member 101b will be described. FIGS. 29 and 31 are perspective views showing the mechanism for preventing dropping of the tube, and FIGS. 30 and 32 are cross sectional views of the mechanism for preventing dropping of the tube.

Referring to FIGS. 29 and 30, at a tip end portion of second pinching member 101b, a lever 101e is provided rotatable about an axis of rotation 101f. At a lower end of lever 101e, a coil spring 101g is attached, and by the energizing force of coil spring 101g, one end of lever 101e is forced to abut a tip end portion of first pinching member 101a. Therefore, in a normal state, the space between the first and second pinching members 101a and 101b is closed. As to the operation, when the other end of lever 101e is pushed down (F1) by a finger or the like, the space between the first and second pinching members 101a and 101b is opened, allowing attachment/detachment of contrast medium introducing tube 3000.

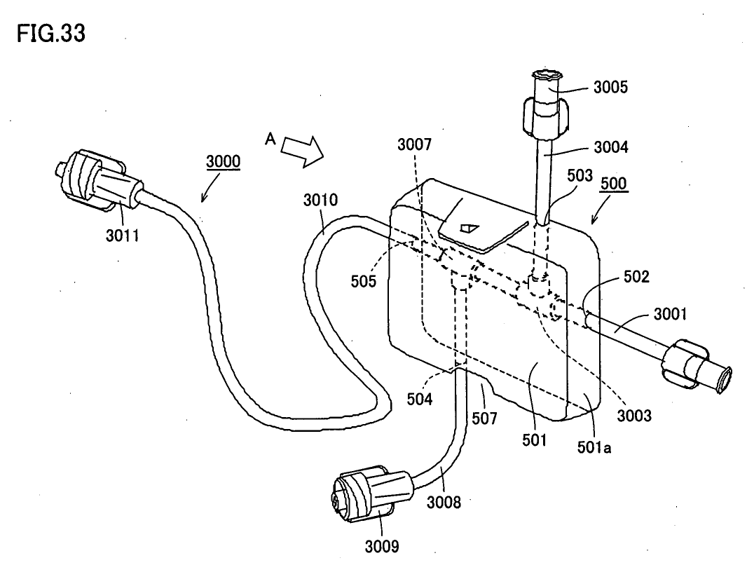

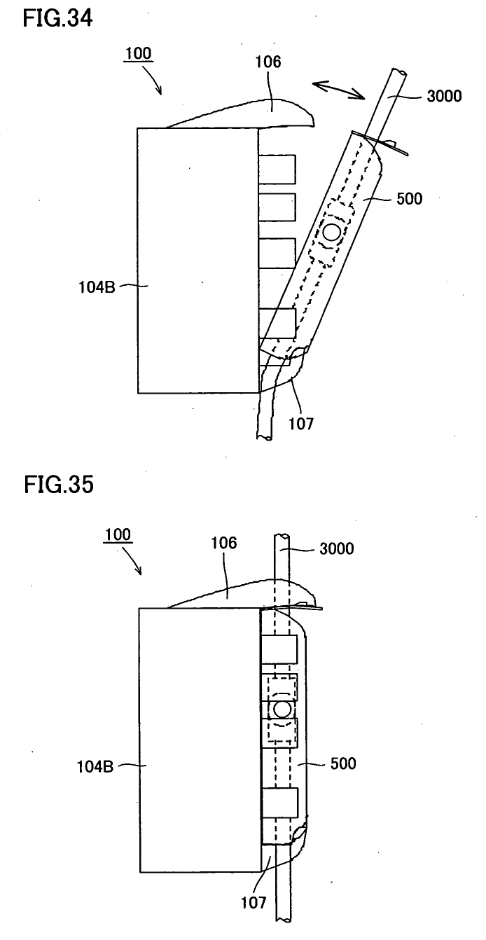

As another mechanism for preventing dropping of the tube, a structure such as shown in FIGS. 33 to 35 may be used. FIG. 33 is a perspective view showing an overall structure of a cassette 500, which will be described later, and FIGS. 34 and 35 are schematic illustrations showing how cassette 500 is attached to main board 104B, viewed from the direction of the arrow A in FIG. 33.

Referring to FIG. 33, the mechanism for preventing dropping of the tube employs a cassette 500 as a shape holding member, for holding contrast medium introducing tube 3000 in a state to be mounted to main board 104B of flow path switching apparatus 100 having the same structure as the flow path switching apparatuses described above. Cassette 500 is attachable/detachable to and from the main board 104B, and in order to hold contrast medium introducing tube 3000 in a state to be mounted to main board 104B, a first engaging portion 502 to be engaged with the first main tube 3001, a second engaging portion 503 to be engaged with the first branch tube 3004, a third engaging portion 504 to be engaged with the second branch tube 3008 and a fourth engaging portion 505 to be engaged with the third main tube 3010 are formed on a side surface portion 501a as side wall of cassette body 501.

Further, on upper and lower side surfaces of side surface portion 501a, an engaging plate 506 and an engaging recess 507 are formed, to be engaged with engaging portions 106 and 107 (see FIG. 34) of main board 104B.

Cassette 500 having the above described structure is mounted on main board 104B in the following manner. As shown in FIG. 34, contrast medium introducing tube 3000 is fixed in cassette 500, and engaging plate 506 of cassette 500 is fitted to engaging portion 106 of main board 104B, with the engaging recess 507 of cassette 500 being engaged with engaging portion 106 of main board 104B (see FIG. 35).

As contrast medium introducing tube 3000 is held in advance in a state ready to be mounted to main board 104B of flow path switching apparatus 100A, it becomes possible to mount contrast medium introducing tube 3000 easily and correctly to flow path switching apparatus 100A by one operation (one touch).

There would be no error in the position of contrast medium introducing tube 3000 for mounting to flow path switching apparatus 100A, and therefore, error in manual operation can be avoided.

Even when contrast medium introducing tube should be damaged and contrast medium 2000 should be discharged, cassette 500 prevents scattering of contrast medium 2000, and therefore, damage to the patient, operator or medical equipment may be avoided.

In each of the embodiments above, a pair of cylindrical first and second pinching members 101a and 101b, pinching from the outside the tube path of the first branch tube 3004 is provided for deforming the tube wall of first branch tube 3004, a pair of cylindrical third and fourth pinching members 102a and 102b, pinching from the outside the tube path of second main tube 3006 is provided for deforming the tube wall of second main tube 3006, and a pair of cylindrical fifth and sixth pinching members 103a and 103b pinching from the outside the tube path of second branch tube 3008 is provided for deforming tube wall of second branch tube 3008. Means for deforming tube wall of each tube is not limited to a mechanism that pinches the tube path from the outside, and mechanism of bending each tube or squeezing each tube may be employed.

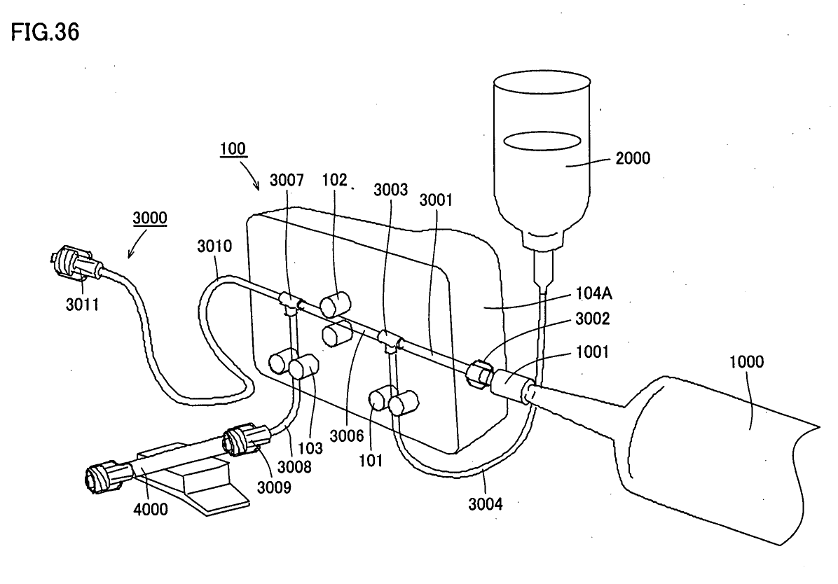

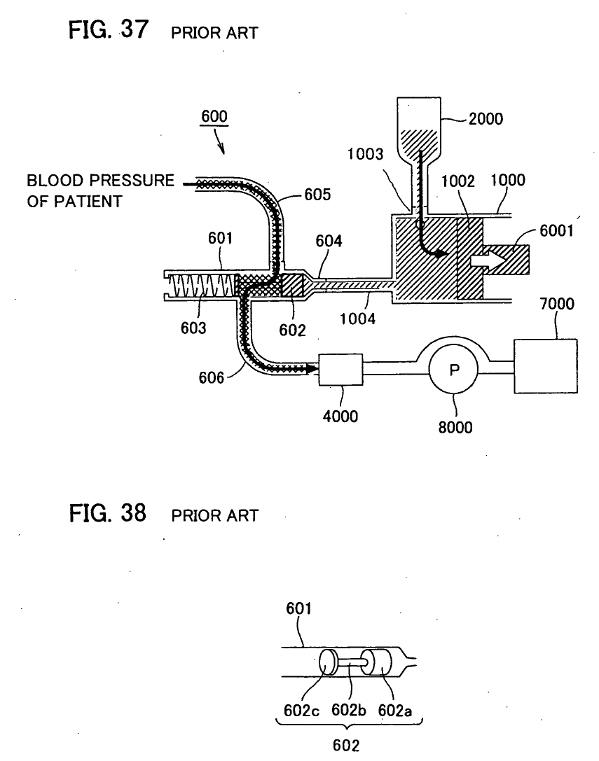

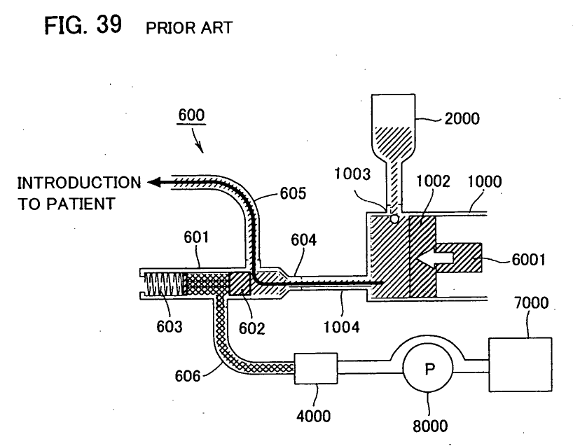

In the description of flow path switching apparatuses 100, 100A, 200, 300 and 400 as well as contrast medium introducing tube 3000, the first branch tube 3004 has been described as arranged upward and the second branch tube 3008 arranged downward. The arrangement is not limited thereto, and, by way of example, first and second branch tubes 3004 and 3008 may be arranged downward as shown in FIG. 36.

In the mechanism of the second to fourth embodiments above, considering possible failure of sliding or rotation of driving plate caused by malfunction of the apparatus, a manual driving means may be provided.

Although the present invention has been described and illustrated in detail, it is clearly understood that the same is by way of illustration and example only and is not to be taken by way of limitation, the spirit and scope of the present invention being limited only by the terms of the appended claims.

### Industrial Applicability

The flow path switching apparatus in accordance with the present invention allows use of a contrast medium introducing tube having a structure that includes only the main tube, the first branch tube and the second branch tube. Therefore, the structure of the contrast medium introducing tube can be simplified. As a result, use of the custom-made piston having the double structure becomes unnecessary, and the cost of the contrast medium introducing tube can be reduced. Further, as the structure of the contrast medium introducing tube is simplified, air trapping in the contrast medium introducing tube can also be reduced.

The contrast medium introducing tube in accordance with the present invention eliminates the use of the custom-made piston having the double structure, and therefore, the cost of the contrast medium introducing tube can significantly be reduced. Further, as the structure of the contrast medium introducing tube is simplified, air trapping in the contrast medium introducing tube can also be reduced.

1. A flow path switching apparatus to be used with a contrast medium introducing tube including a main tube having one end coupled to an outlet port of a syringe to be filled with a contrast medium and the other end coupled to a patient side, a first branch tube branched from the main tube and coupled to a contrast medium reserving member, and a second branch tube branching from said main tube at a portion closer to the other end than the first branch tube and coupled to a pressure transducer and to a physiological saline reserving member storing physiological saline, comprising:
first branch tube opening/closing means for closing flow path of said first branch tube by deforming tube wall of said first branch tube and for opening the flow path of said first branch tube by recovering the tube wall of said first branch tube;
main tube opening/closing means arranged between said first branch tube and said second branch tube for closing flow path of said main tube by deforming tube wall of said main tube and for opening the flow path of said main tube by recovering the tube wall of said main tube; and
second branch tube opening/closing means for closing flow path of said second branch tube by deforming tube wall of said second branch tube and for opening the flow path of said second branch tube by recovering the tube wall of said second branch tube.

2. The flow path switching apparatus according to claim 1, wherein
said first branch tube opening/closing means includes first pinching means for pinching said tube path from outside to deform the tube wall of said first branch tube;
said main tube opening/closing means includes main tube pinching means for pinching said tube path from outside to deform tube wall of said main tube; and
said second branch tube opening/closing means includes second pinching means for pinching said tube path from outside to deform tube wall of said second branch tube.

3. The flow path switching apparatus according to claim 2, further comprising
switching means for selecting:
a first state in which flow paths of said first branch tube and said second branch tube are opened by said first pinching means and said second pinching means, when said main tube is pinched by said main pinching means and the flow path of said main tube is closed; and
a second state in which flow paths of said first branch tube and said second branch tube are closed by said first pinching means and said second pinching means, when the flow path of said main tube is opened by said main pinching means.

4. The flow path switching means according to claim 3, wherein
said switching means controls said main pinching means, said first pinching means and said second pinching means separately and independently from each other.

5. The flow path switching means according to claim 3, wherein
said switching means has a link mechanism for controlling said main pinching means, said first pinching means and said second pinching means linked with each other.

6. The flow path switching means according to claim 1, further comprising
syringe holding state switching means for switching between a state in which said outlet port of said syringe is held facing approximately upward and a state in which said outlet port of said syringe is held inclined downward, with said main tube held in an approximately horizontal state.

7. A contrast medium introducing tube mounted to a flow path switching apparatus including
a main tube having one end coupled to an outlet port of a syringe to be filled with a contrast medium and the other end coupled to a patient side, a first branch tube branched from the main tube and coupled to a contrast medium reserving member, and a second branch tube branching from said main tube at a portion closer to the other end than the first branch tube and coupled to a pressure transducer and to a physiological saline reserving member storing physiological saline, including
first branch tube opening/closing means for closing flow path of said first branch tube by deforming tube wall of said first branch tube and for opening the flow path of said first branch tube by recovering the tube wall of said first branch tube,
main tube opening/closing means arranged between said first branch tube and said second branch tube for closing flow path of said main tube by deforming tube wall of said main tube and for opening the flow path of said main tube by recovering the tube wall of said main tube, and
second branch tube opening/closing means for closing flow path of said second branch tube by deforming tube wall of said second branch tube and for opening the flow path of said second branch tube by recovering the tube wall of said second branch tube;
said contrast medium introducing tube comprising:
a main tube having one end coupled to a syringe to be filled with a contrast medium and the other end coupled to a patient side;
a first branch tube branched from the main tube and coupled to a contrast medium reserving member storing the contrast medium; and
a second branch tube branching from said main tube at a portion closer to the other end than the first branch tube and coupled to a physiological saline reserving member storing physiological saline.

8. The contrast medium introducing tube according to claim 7, further comprising
a shape holding member for holding said contrast medium introducing tube in a state to be mounted to said flow path switching apparatus.

9. The contrast medium introducing tube according to claim 8, wherein
said shape holding member is attachable to/detachable from said flow path switching means.
